(19) United States
(12) Patent Application Publication
(10) Pub. No.: US 2018/0149583 A1
(43) Pub. Date: May 31, 2018

(54) METHOD AND APPARATUS FOR PROVIDING FOOD INFORMATION

(71) Applicant: Do Yeon PI, Seoul (KR)

(72) Inventor: Do Yeon PI, Seoul (KR)

(21) Appl. No.: 15/879,733

(22) Filed: Jan. 25, 2018

Related U.S. Application Data
(63) Continuation of application No. PCT/KR2016/008295, filed on Jul. 28, 2016.

(30) Foreign Application Priority Data
Jul. 29, 2015 (KR) 10-2015-0107090

(51) Int. Cl. G01N 21/17; G09B 19/00; G16H 20/60; G01N 33/02

(52) U.S. Cl. CPC G01N 21/171; G01N 33/02; G16H 20/60; G09B 19/0092

(57) ABSTRACT

Disclosed are a method and an apparatus for providing food information, by which the kind, the total calorie, and the suitable intake calorie of a food which the user is to take may be conveniently identified. A food information providing apparatus includes an optical spectrum obtaining unit configured to obtain an optical spectrum of a food which a user is to take, a depth image obtaining unit configured to obtain a depth image of the food, and a controller configured to identify a kind of the food based on the optical spectrum of the food, wherein the controller sets a figure with reference to a food area detected from the depth image of the food, and calculates at least one of a volume, a total calorie, and a suitable intake calorie of the identified food based on the set figure.

### CROSS-REFERENCE TO RELATED APPLICATIONS

The present application is a continuation of International Patent Application No. PCT/KR2016/008295, filed on Jul. 28, 2016, which is based upon and claims the benefit of priority to Korean Patent Application No. 10-2015-0107090, filed on Jul. 29, 2015. The disclosures of the above-listed applications are hereby incorporated by reference herein in their entirety.

### BACKGROUND

Embodiments of the inventive concept described herein relate a method and an apparatus for providing food information. More particularly, the inventive concept relates to a method and an apparatus for providing food information, which may help the user maintain or improve the health of the user by providing the user with food information before or after meal.

People take various kinds of foods in everyday lives. Taking a food of a suitable calorie is helpful to the health, but taking a calorie that is more than a recommended daily calorie is not good for health.

However, it is not easy to identify and manage how much the calorie of the food which a person is to take is and how much the suitable intake calorie is.

### SUMMARY

Embodiments of the inventive concept provide a method and an apparatus for providing food information, by which the kind, the total calorie, and the suitable intake calorie of a food which the user is to take may be conveniently identified.

In accordance with an aspect of the inventive concept, there is provided a food information providing apparatus including an optical spectrum obtaining unit configured to obtain an optical spectrum of a food which a user is to take, a depth image obtaining unit configured to obtain a depth image of the food, and a controller configured to identify a kind of the food based on the optical spectrum of the food, wherein the controller sets a figure with reference to a food area detected from the depth image of the food, and calculates at least one of a volume, a total calorie, and a suitable intake calorie of the identified food based on the set figure.

The controller may convert image coordinates of pixels included in the detected food area to world coordinates, and may set one or more figures such that the one or more figures include the food area converted to the world coordinates.

The controller may convert image coordinates of pixels included in the detected food area to world coordinates, and may set one or more figures in the food area converted to the world coordinates.

The figures may include three-dimensional figures including at least one of a sphere, a cone, a cylinder, and a hexahedron.

The food information providing apparatus may further include a storage configured to store a food information table, and the food information table may include at least one of kinds of foods, types of natural optical spectrums of the foods, and calories per unit volume of the foods.

The controller may obtain the calorie per unit volume of the identified food from the food information table, and may calculate a total calorie of the identified food based on the calorie per unit volume of the identified food and the volume of the identified food.

The controller may calculate a suitable intake calorie for the identified food with reference to at least one of current body information of the user and body information that is targeted by the user.

The food information providing apparatus may further include an output unit configured to output at least one of a kind of the identified food, a total calorie of the identified food, and a suitable intake calorie of the identified food in at least one form including a letter, an image, and a voice.

The controller may detect a food area corresponding to the food from the image obtained by photographing the food, and may highlight and displays an area of the detected food area, which corresponds to the suitable intake calorie.

In accordance with another embodiment of the inventive concept, there is provided a method for providing food information, the method including obtaining an optical spectrum of a food which a user is to take, obtaining a depth image of the food, identifying a kind of the food based on the optical spectrum of the food, and setting a figure with reference to a food area detected from the depth image of the food, and calculating at least one of a volume, a total calorie, and a suitable intake calorie of the identified food based on the set figure.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a view illustrating a configuration of a food information providing apparatus according to an embodiment of the inventive concept;

FIG. 2 is a view exemplifying that foods are photographed by using the food information providing apparatus of FIG. 1;

FIG. 3 is a view exemplifying optical spectrums for foods measured by using the food information providing apparatus of FIG. 1;

FIG. 4 is a view for explaining a process of identifying the kind of a food based on an optical spectrum measured by using the food information providing apparatus of FIG. 1;

FIGS. 5 to 8 are views for explaining a process of calculating a total calorie of a food and a suitable intake calorie of the food based on an image captured by using the food information providing apparatus of FIG. 1, and a scheme of displaying the calculated information; and

FIG. 9 is a flowchart illustrating a method for providing food information according to an embodiment of the inventive concept.

### DETAILED DESCRIPTION

The above and other aspects, features and advantages of the invention will become apparent from the following description of the following embodiments given in conjunction with the accompanying drawings. However, the inventive concept is not limited to the embodiments disclosed below, but may be implemented in various forms. The embodiments of the inventive concept is provided to make the disclosure of the inventive concept complete and fully inform those skilled in the art to which the inventive concept pertains of the scope of the inventive concept.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

The terms used herein are provided to describe the embodiments but not to limit the inventive concept. In the specification, the singular forms include plural forms unless particularly mentioned. The terms “comprises” and/or “comprising” used herein does not exclude presence or addition of one or more other elements, in addition to the aforementioned elements.

Hereinafter, exemplary embodiments of the inventive concept will be described with reference to the accompanying drawings. In the drawings, the same reference numerals denote the same elements.

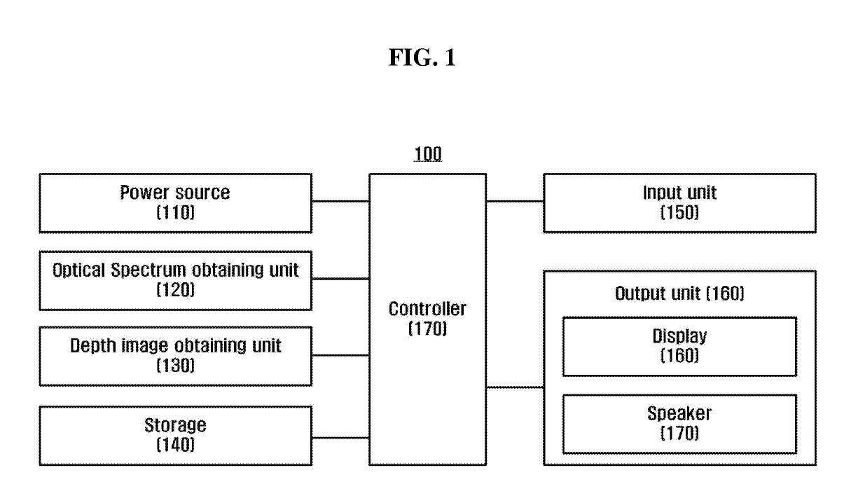

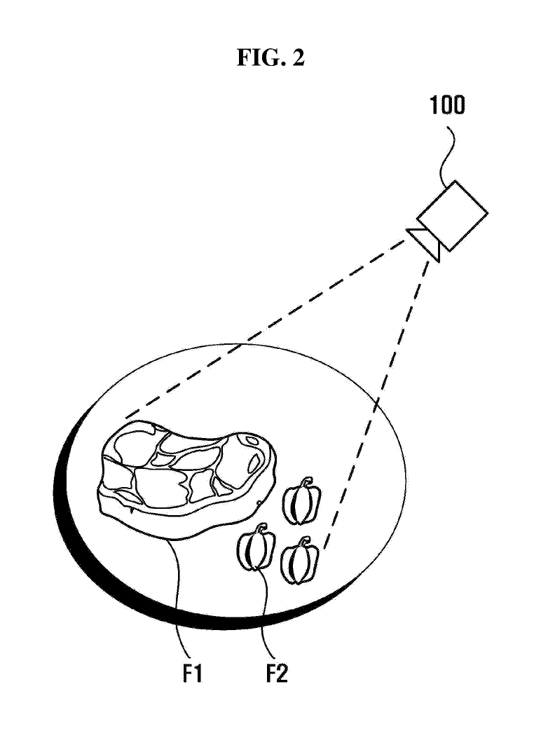

FIG. 1 is a view illustrating a configuration of a food information providing apparatus 100 according to an embodiment of the inventive concept. FIG. 2 is a view exemplifying that foods are photographed by using the food information providing apparatus 100 according to the embodiment of the inventive concept.

Referring to FIG. 1, the food information providing apparatus 100 according to the embodiment includes a power source 110, an optical spectrum obtaining unit 120, a depth image obtaining unit 130, a storage 140, an input unit 150, an output unit 160, and a controller 170.

The power source 110 supplies electric power to the elements of the food information providing apparatus 100. According to an embodiment, the power source 110 may be mechanically and electrically separated from the food information providing apparatus 100. The separated power source 110 may be exchanged with another marginal power source (not illustrated). According to an embodiment, the power source 110 may be integrally formed with the food information providing apparatus 100. In this case, the power source 110 may receive electric power from a separately provided charging device (not illustrated) to be charged. Then, the power source 110 may receive electric power from the charging device according to a wired power transmission technology or a wireless power transmission technology. In the latter case, the charging device detects whether the food information providing apparatus 100 is positioned on the charging device, and when it is detected that the food information providing apparatus 100 is positioned on the charging device, supplies electric power to the power source 110 of the food information providing apparatus 100 according to the wireless power transmission technology. The wireless power transmission technology may be classified into a magnetic induction (MI) scheme, a magnetic resonant (MR) scheme, and a microwave radiation scheme, the power source 110 may wirelessly receive electric power according to one of the exemplified schemes.

The optical spectrum obtaining unit 120 photographs foods F1 and F2 and obtains optical spectrums for the foods F1 and F2. In detail, the optical spectrum obtaining unit 120 photographs and obtains images of the foods F1 and F2 or reflective light reflected from the foods F1 and F2, and may obtain an optical spectrum on the corresponding foods F1 and F2.

The depth image obtaining unit 130 obtains a depth image for a food. In order to obtain a depth image, microwaves, light waves, and ultrasonic waves may be used. The scheme that uses light waves, for example, may include a triangulation method, a time-of-flight method, and an interferometry method. The depth image obtaining unit 130 may obtain depth images for the foods F1 and F2 by using one of the exemplified methods.

Based on the triangulation method, the depth image obtaining unit 130 obtains images from two cameras (hereinafter, a ‘stereo camera’) having specific baselines that is like the two eyes of a human being, and finds corresponding points in two images and obtains a depth image.

Meanwhile, one camera of the stereo camera may be replaced by a pattern projector that may project a pattern. The pattern projector irradiates light of a predefined pattern, that is, structured light to a surface of an object (for example, a food). The structured light irradiated to the surface of the object is distorted by curves on the surface of the object. The structured light distorted by the surface of the object is photographed by a camera disposed at a location that is different from the location of the pattern projector. As the structured light irradiated from the pattern projector and the structured light distorted by the curved on the surface of the object are compared, a depth image of the object may be obtained.

Based on the time-of-flight method, the depth image obtaining unit 130 measures a time period for a specific light wave returns after the light wave is irradiated to an object, and obtains a depth image for the object. To achieve this, the depth image obtaining unit 130, for example, may include a TOF sensor. The TOF sensor may include a signal transmitting unit that transmits light modulated to a signal of a specific frequency, and a signal receiving unit that receives the light that is reflected by the object and then returns.

The storage 140 may include a nonvolatile memory, a volatile memory, an embedded memory, a detachable external memory, a hard disk, an optical disk, a magneto-optical disk, or an arbitrary computer-readable recording medium known in the art to which the inventive concept pertains. The external memory, for example, may include a secure digital (SD) card, a mini-SD card, and a micro-SD card.

The storage 140 stores at least one of data, software, and an application that is necessary for the food information providing apparatus 100 to be operated. For example, the storage 140 stores a food information database. The food information database may include the kinds of foods, natural optical spectrum information for foods, and information on calories per unit volume of foods.

The food information database may be renewed continuously. For example, the optical spectrum information on a new food and information of a calorie per unit volume of the new food may be downloaded to the storage 140 through a wired/wireless network. As another example, the food information database may be renewed by exchanging a detachable disk in which an existing food information database is stored with a detachable disk in which a new food information database is stored.

In addition, the storage 140 may store user information. Identification information and body information may be exemplified as user information. The identification information refers to information for identifying a user, and for example, may include a name, an ID, and a password. The body information refers to various pieces of information on the body of the user, and for example, may include a sex, an age, a height, a weight, lengths of parts of the body, and circumferences of parts of the body. The exemplified user information may be directly input by the user, may be received from another device (not illustrated) through a wired/wireless network, or may be detected from an image obtained by photographing a user.

The input unit 150 receives various pieces of information from the user. To achieve this, the input unit 150 may include a touchpad, a keypad, a button, a switch, a jog wheel, or an input unit including a combination thereof. The touch pad may be stacked on a display 161 of the output unit 160, which will be described below, to constitute a touchscreen.

The output unit 160 outputs a command processing result and various pieces of information to the user. For example, the output unit 160 outputs food information of the foods F1 and F2 which the user is to take. The food information, for example, may include the kind of a food which the user is to take, a total calorie of the food, and a suitable intake calorie. The exemplified food information may be output in at least one form of a letter, an image, and a voice. To achieve this, the output unit 160 may include a display 161 and a speaker 162. The display 161 may be a flat panel display, a flexible display, an opaque display, a transparent display, an electronic paper (E-paper), or an arbitrary form known in the art to which the inventive concept pertains. In addition to the display 161 and the speaker 162, the output unit 160 may further include an arbitrary form of output unit that is well known in the art to which the inventive concept pertains.

The controller 170 connects and controls other elements in the food information providing apparatus 100. For example, the controller 170 compares the optical spectrum information obtained by the optical spectrum obtaining unit 120 and the optical spectrum information stored in the food information database, and identifies the kind of the food which the user is to take. The identified kind of the food may be output in a form of a letter, an image, a voice, or a combination thereof.

As another example, the controller 170 calculates a total calorie of the food which the user is to take. In detail, if a depth image of the food is obtained by the depth image obtaining unit 130, the controller 170 detects a food area from the depth image and sets a figure corresponding to the detected food area. Thereafter, the controller 170 calculates the volume of the food area based on the set figure. Next, the controller 170 searches the food information database stored in the storage 140, and obtains information on a calorie per unit volume of the corresponding food. Further, the total calorie of the corresponding food is calculated by multiplying the information of the calorie per unit volume and the calculated volume. The calculated total calorie may be output in a form of a letter, an image, a voice, or a combination thereof.

As another example, the controller 170 calculates a suitable intake calorie of the food which the user is to take. The suitable intake calorie for the corresponding food may be calculated based on current body information of the user and/or the body information that is targeted by the user. As an example, when the user desires to maintain the body information as now, the controller 170 calculates a recommended daily allowance calorie based on the current body information of the user. Further, the suitable intake calorie for the food which the user is to take is calculated based on the recommended daily allowance calorie and the calorie that has been taken by the user until now. The calculated daily allowance calorie may be output in a form of a letter, an image, a voice, or a combination thereof.

The controller 170 outputs the above-described food information in a form of a letter, an image, a voice, or a combination thereof. For example, after calculating a suitable intake calorie for the specific food, the controller 170 may calculate a volume corresponding to the calculated suitable intake calorie and may display the food area corresponding to the calculated volume through the display 161 while highlighting the food area.

Meanwhile, the functional blocks illustrated in FIG. 1 are simply examples for explaining the embodiment of the food information providing apparatus 100 of the inventive concept, and some of the functional blocks illustrated in FIG. 1 may be omitted or a new functional block that is not illustrated in FIG. 1 may be added to the food information providing apparatus 100 of the inventive concept. For example, the food information providing apparatus 100 may further include a color image obtaining unit (not illustrated) that obtains color images for the foods F1 and F2, in addition to the elements illustrated in FIG. 1. The color image obtaining unit, for example, may include a charge coupled device (CCDE) image sensor or a complementary metal oxide semiconductor (CMOS) image sensor.

The above-described food information providing apparatus 100 may include a wired/wireless communication device. The wired/wireless communication device, for example, may include a personal computer (PC), a cellular phone, a personal communication service (PCS) phone, a synchronous/asynchronous international mobile telecommunication-2000 (IMT-2000) mobile terminal, a palm personal computer (PC), a personal digital assistant (PDA), a smartphone, a tablet, a wireless application protocol (WAP) phone, and a mobile gaming device. The exemplified digital device may be a wearable device that may be mounted on the body of the user.

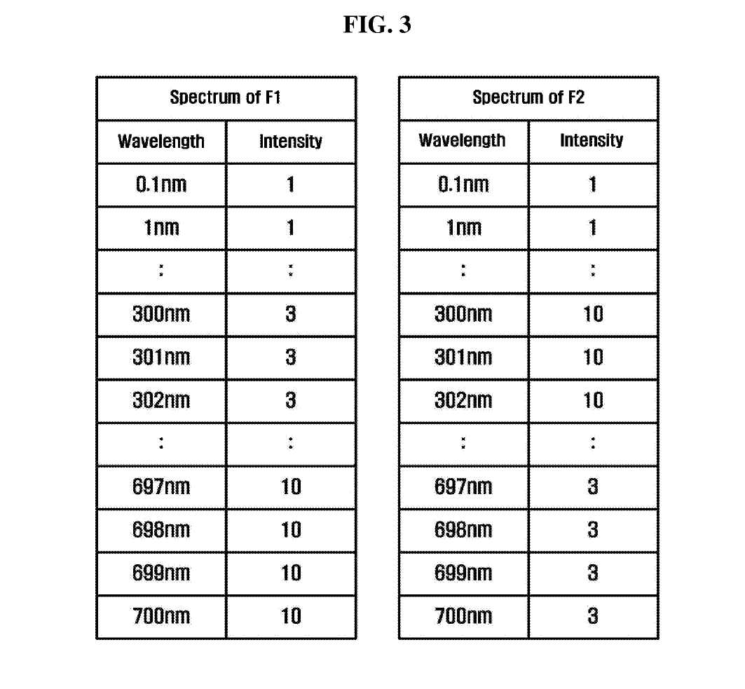

FIG. 3 is a view exemplifying optical spectrums for foods measured by using the food information providing apparatus 100 of FIG. 1.

Referring to FIG. 3, it can be seen that the first food F1 and the second food F2 have different optical spectrums. In detail, in the optical spectrum of the first food F1, the intensity of the light of a long wavelength band (exemplified as about 700 nm) is about 10, which is stronger than those of other bands. In the optical spectrum of the second food F2, the intensity of the light of a long wavelength band (exemplified as about 300 nm) is about 10, which is stronger than those of other bands. That is, the foods may have different natural optical spectrums, and the food information providing apparatus 100 may identify the kind of the food by analyzing the optical spectrum of the food.

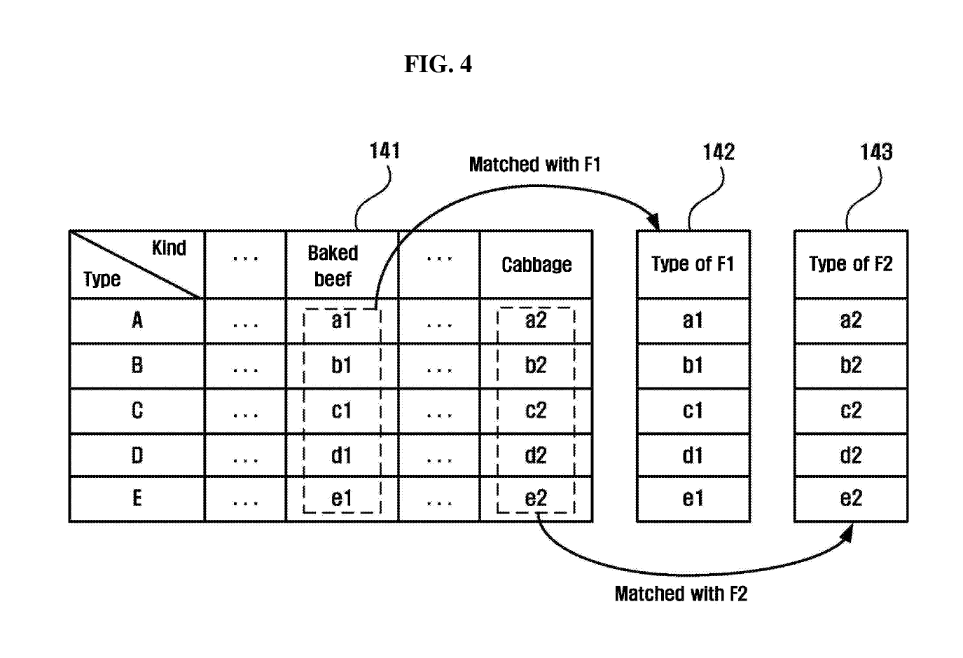

FIG. 4 is a view for explaining a process of identifying the kind of a food based on an optical spectrum measured by using the food information providing apparatus 100 of FIG. 1.

A food information table 141 is exemplified on the left side of FIG. 4. The “kinds” of the foods are listed on the transverse axis of the food information table 141, and the “types” for the kinds of the foods are listed on the longitudinal axis of the food information table 141. Here, the “types” means values that may characterize natural optical spectrums of the foods. For example, the type may include values, such as at which wavelength band of the optical spectrum the intensity of the light is predominant, how the optical spectrum changes as the wavelength increases or decreases, how the entire intensity of the optical spectrum is, or how an average intensity for wavelength bands of the optical spectrum is, but the inventive concept is not limited thereto. In the following, the average intensities of light for wavelength bands of the optical spectrums will be exemplified as values that may characterize the natural optical spectrums of the foods.

Five types (A, B, C, D, and E) are exemplified in the food information table 141 of FIG. 4. Here, type A means an average intensity of light of the shortest wavelength band in an optical spectrum measured for a specific food. Further, type E means an average intensity of light of the longest wavelength band in an optical spectrum measured for a specific food. Type B means an average intensity of light of a band of a wavelength that is longer than that of type A, type C means an average intensity of light of a band of a wavelength that is longer than that of type B, and type D means an average intensity of light of a band of a wavelength that is longer than that of type C. The food information table 141 illustrated in FIG. 4 may be stored in the food information database of the storage 140.

Meanwhile, values for the types of the optical spectrums of the first food F1 and values for the types of the optical spectrums of the second food F2 are exemplified on the right side of FIG. 4. As illustrated in FIG. 4, when the values for the types of the optical spectrums of the first food F1 are ‘a1’, ‘b1’, ‘c1’, ‘d1’, and ‘e1’, they may be compared with and matched with the values of the types of the optical spectrums of ‘baked beef’, among the foods stored in the food information table 141. Accordingly, the controller 170 may identify the kind of the first food F1 as ‘baked beef’.

Similarly, when the values for the types of the optical spectrums of the second food F2 are ‘a2’, ‘b2′, ‘c2’, ‘d2’, and ‘e2’, they may be compared with and matched with the values of the types of the optical spectrums of ‘cabbage’, among the foods stored in the food information table 141. Accordingly, the controller 170 may identify the kind of the second food F2 as ‘cabbage’.

Meanwhile, although not illustrated in FIG. 4, the food information table 141 may further include information of calories per unit volume of the foods.

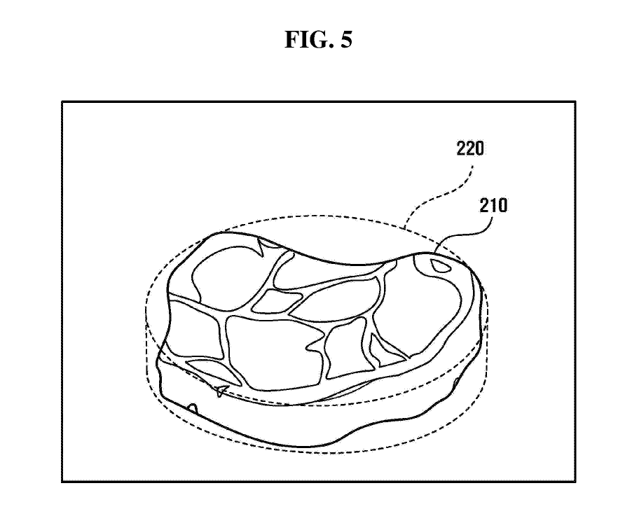

FIGS. 5 to 8 are views for explaining a process of calculating a total calorie 250 of a food and a suitable intake calorie 260 of the food based on an image captured by using the food information providing apparatus 100, and a scheme of displaying the calculated information.

For example, when the user is to take the first food F1, the user photographs the first food F1 by using the food information providing apparatus 100. Then, a depth image for the first food F1 is obtained by the depth image obtaining unit 130. When a color image obtaining unit is additionally provided, a color image for the first food F1 is also obtained. In this case, the color image for the first food F1 may be disposed in real time through the display 161, and the depth image for the first food F1 may be provided to the controller 170 instead of being displayed through the display 161.

After detecting a food area that is an area corresponding to the first food F1 from the obtained depth image, the controller 170 converts the image coordinates of the pixels included in the food area to world coordinates.

Then, the controller 170 sets a figure 220 such that the figure 220 includes the food area 210 converted to the world coordinates. The figure 220 includes a 3-dimensional figure such as a sphere, a cone, a cylinder, and a hexahedron. FIG. 5 illustrates that one cylinder is set as the figure 220 including the food area 210. However, the inventive concept is exemplary and is not necessarily limited thereto. According to another embodiment, a plurality of figures may be set as a figure including the food area 210. Then, the plurality of figures may be similar figures of different sizes, and may be figures having different sizes and shapes. If a plurality of figures are set instead of setting one figure with respect to the food area 210, a total calorie 250 of the first food F1 may be calculated more accurately because a more accurate volume for the food area 210 may be calculated. Hereinafter, for convenience of description, a case in which one cylinder is set with reference to the food area 210 converted to the world coordinates will be described as an example.

As illustrated in FIG. 5, if the figure 220 is set for the food area 210, the controller 170 calculates the volume of the first food F1 based on the set figure 220. For example, the controller 170 calculates the volume of the cylinder 220 based on the area of the bottom surface of the cylinder 220 and the height of the cylinder 220. Then, the calculated volume of the cylinder 220 may be understood as the volume of the first food F1.

In this method, if the volume of the first food F1 is calculated, the controller 170 obtains information on a calorie per unit volume of the first food F1, that is, ‘baked beef 240’ from the food information table 141. Next, the controller 170 calculates the total calorie 250 of the first food F1 by multiplying the volume of the figure 220 calculated in advance and the information on a calorie per unit volume.

If the total calorie 250 of the first food F1 is calculated, the controller 170 calculates a suitable intake calorie 260 for the first food F1 based on current body information of the user and/or body information that is targeted by the user. In detail, when the user desires to maintain the current weight, the controller 170 calculates a remaining intake calorie by calculating a recommended daily calorie that is suitable for the user and subtracting the calorie which has been taken until now from the recommended daily calorie. Further, a suitable intake calorie 260 for the first food F1 is calculated by comparing the remaining intake calorie and the total calorie 250 of the first food F1.

The food information including the kind of the first food F1 identified by the controller 170, the total calorie 250 of the first food F1 calculated by the controller 170, and the suitable intake calorie 260 of the first food F1 may be output through a letter, an image, a voice, or a combination thereof. The kind of the food information that is to be output and/or the output scheme of the food information may be set by the user in advance. Further, the set value may be changed while the food information is being output.

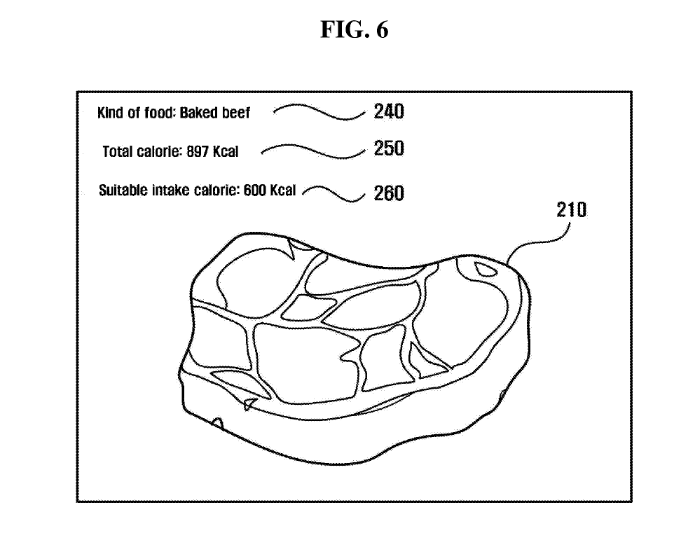

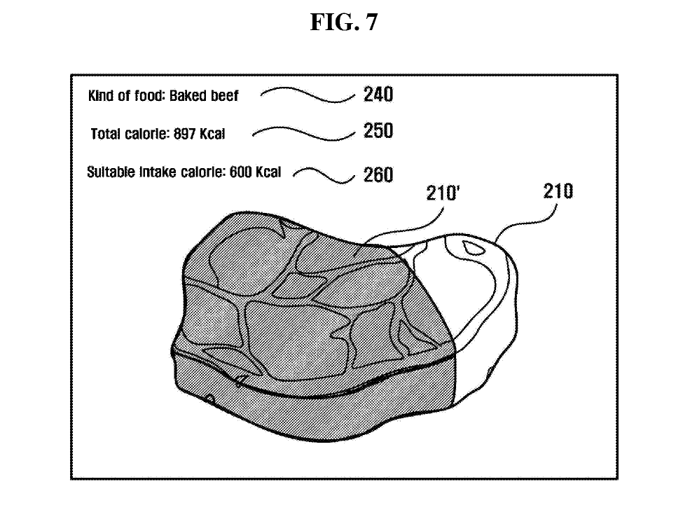

FIG. 6 illustrate that the food information, such as the kind, the total calorie 250, and the suitable intake calorie 260 of the food is all displayed with letters, together with the captured image of the first food F1. FIG. 7 illustrate that the food information, such as the kind, the total calorie 250, and the suitable intake calorie 260 of the food is all displayed with letters, together with the captured image of the first food F1 and the suitable intake calorie 260 is expressed in graphics.

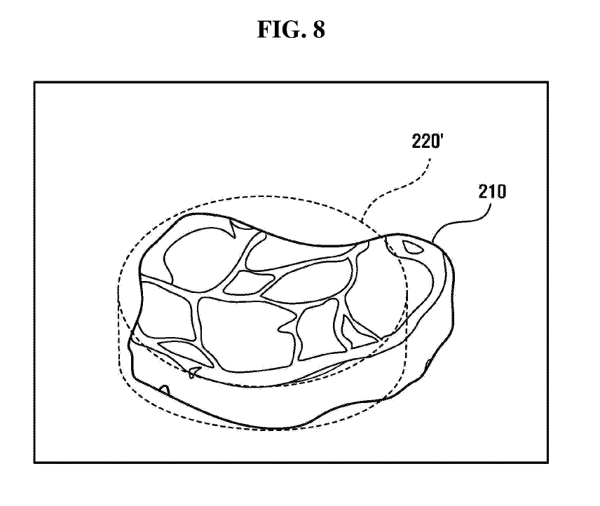

As illustrated in FIG. 7, in order to express the suitable intake calorie 260 in graphics, the controller 170 converts the suitable intake calorie 260 for the first food F1 to a volume. Next, the controller 170 calculates the diameter (or height) of a cylinder by which the calculated volume may be derived. Next, the controller 170 adjusts the size of the cylinder 220 that is set to include the food area 210 based on the calculated diameter (or height). FIG. 8 illustrates a cylinder 220′, the size of which has been adjusted. If FIG. 8 and FIG. 5 are compared, it can be seen that the diameter of the cylinder 220′ of FIG. 8 is reduced as compared with the diameter of the cylinder 220 of FIG. 5. Thereafter, the controller 170 highlights the food area 210 included in the cylinder 220′, the size of which has been adjusted. As a result, as illustrated in FIG. 7, a suitable intake calorie 260 of the first food F1 may be expressed in graphics. As illustrated in FIG. 7, if the suitable intake calorie 260 of the first food F1 is disposed to overlap the food area in the image, the user may intuitively recognize the suitable intake calorie 260 for the first food F1. Further, because the user has meal while recognizing the suitable intake calorie 260 for the first food F1, the user may decrease a probability of excessively taking the first food F1.

Until now, the configuration of the food information providing apparatus 100, the process of obtaining food information by the food information providing apparatus 100, and the method for outputting the obtained food information according to the embodiment of the inventive concept have been described with reference to FIGS. 1 to 8. It has been exemplified in the above-described embodiment that if the user photographs a food before meal by using the food information providing apparatus 100, the food information of the corresponding food is also displayed in the captured image.

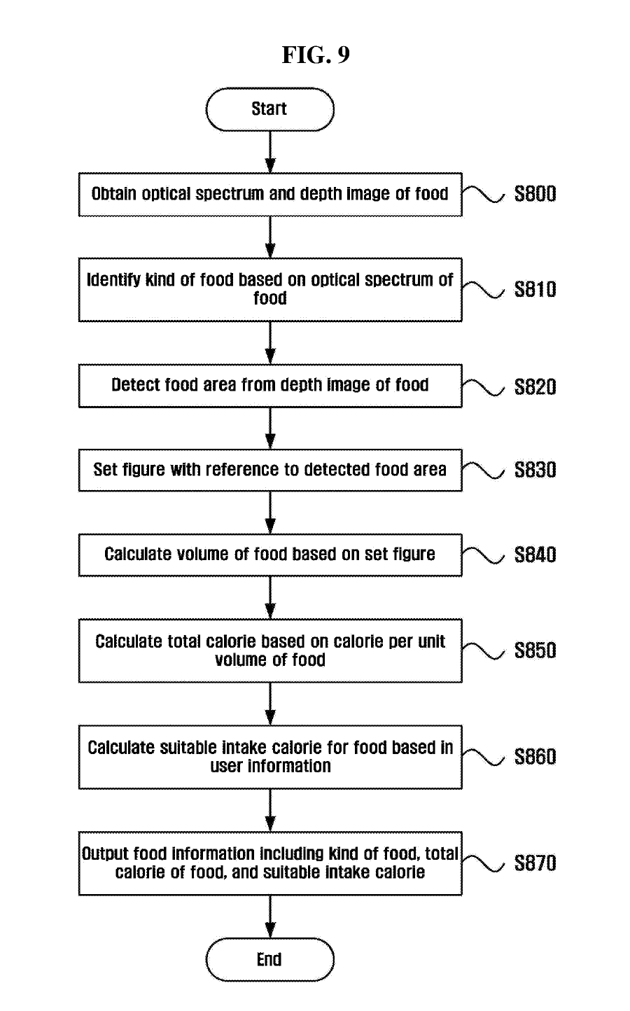

According to another embodiment, the user may photograph the corresponding food even after meal by using the food information providing apparatus 100. In this case, the food information providing apparatus 100 may detect a food area from the image captured after meal, and may calculate the detected volume of the food area. Further, the volume that is actually taken by the user is calculated by subtracting the volume of the food area calculated based on the image captured before meal from the volume of the food area calculated based on the image captured after meal. Further, an actual intake calorie that has been actually taken is calculated by multiplying the calculated volume by the calorie per unit volume. The calculated actual intake calorie may be stored in the storage 140. The actual intake calorie stored in the storage 140 may be summed in a specific unit. For example, the actual intake calorie may be summed in unit of a day, a week, or a month. FIG. 9 is a flowchart illustrating a method for providing food information according to an embodiment of the inventive concept.

Prior to description, it is assumed that identification information of the user, current body information of the user, body information that is targeted by the user, and information on a calorie that has been taken by the user until now are stored in the storage 140 of the food information providing apparatus 100. Further, it is assumed that the kind of the food information that is to be output and the output scheme of the food information are set.

First, an optical spectrum and a depth image of the food that is to be taken by the user are obtained (S800). The optical spectrum of the food is obtained by the optical spectrum obtaining unit 120, and the depth image is obtained by the depth image obtaining unit 130. In operation S800, a color image of the food together with the optical spectrum and the depth image may be obtained.

Thereafter, the kind of the food is identified based on the optical spectrum of the food (S810). Operation S810 includes an operation of comparing the type of the obtained optical spectrum and the type of the optical spectrum stored in the food information table 141, and an operation of identifying the kind of the food which the user is to take based on the comparison result.

Meanwhile, a food area that is an area corresponding to the food is detected from the depth image of the food (S820).

Thereafter, one or more figures are set with reference to the detected food area (S830). According to an embodiment, operation S830 may include an operation of converting image coordinates of the pixels included in the detected food area to world coordinates, and an operation of setting one or more figures such that the figures include the food area converted to the world coordinates. According to another embodiment, operation S830 may include an operation of converting image coordinates of the pixels included in the detected food area to world coordinates, and an operation of setting one or more figures in the food area converted to the world coordinates. Here, the figures may mean three-dimensional figures such as a sphere, a cone, a cylinder, and a hexahedron.

After operation S830, the volume of the food area is calculated based on the one or more set figures (S840). The volume of the food area calculated in operation S840 may be understood to be the actual volume of the identified food.

Thereafter, the total calorie of the identified food is calculated based on the calorie per unit volume of the identified food (S850). Operation S850 may include an operation of calculating a total calorie of the identified food by multiplying the calorie per unit volume of the identified food and the actual volume of the identified food. The calorie of unit volume of the identified food is obtained from the food information table 141 exemplified in FIG. 4.

Thereafter, a suitable intake calorie for the identified food is calculated based on the user information (S860). Operation S860 includes an operation of calculating a recommended daily calorie that is suitable for the user based on current body information of the user and/or body information that is targeted by the user, an operation of calculating a remaining intake calorie by subtracting a calorie that has been taken until now by the user from the recommended daily calorie, and an operation of calculating a suitable intake calorie for the identified food by comparing the remaining intake calorie and the total calorie of the identified food.

Thereafter, the food information including at least one of the kind of the identified food, the total calorie of the identified food, and the suitable intake calorie is output through the output unit 160 (S870). Operation S870 includes an operation of expressing the suitable intake calorie in graphics. The operation of expressing the suitable intake calorie in graphics may include an operation of converting the suitable intake calorie for the identified food to a volume, an operation of calculating a parameter (for example, a diameter or a height) of the figure by which the converted volume is derived, an operation of adjusting the size of the figure that is set to include a food area based on the calculated parameter, and an operation of highlighting a food area included in the figure, the size of which has been adjusted.

Until now, the embodiments of the inventive concept have been described. In addition to the above-described embodiments, the embodiments of the inventive concept may be implemented through a medium that includes a computer readable code/command for controlling at least one processing element of the above-described embodiments, for example, a computer readable medium. The medium may correspond to a medium (s) that allows storage and/or transmission of the computer readable code.

The computer readable code may not only be recorded in a medium but also be transmitted through the internet, and the medium may, for example, include a recording medium such as a magnetic storage medium (for example, an ROM, a floppy disk, or a hard disk) and an optical recording medium (for example, a CD-ROM, Blu-Ray, or a DVD), or a transmission medium such as carrier waves. Because the mediums may correspond to a distribution network, the computer readable code may be stored, transmitted, and executed in a distribution scheme. Moreover, simply as an example, the processing element may include a processor or a computer processor, and the processing element may be distributed and/or included in one device.

If a food which the user is to take is photographed, the food information such as the kind, the total calorie, and the suitable intake calorie of the corresponding food is provided to the user so that the user may be informed before the food is taken and the user may be prevented from excessively taking the food.

Because the suitable intake calorie of the food which the user is to take is calculated based on the user information and the calculated suitable intake calorie is visually displayed to overlap the food area in the image, the user may intuitively recognize the suitable intake calorie for the corresponding food.

Although the exemplary embodiments of the inventive concept have been described with reference to the accompanying drawings, it will be understood by those skilled in the art to which the inventive concept pertains that the inventive concept can be carried out in other detailed forms without changing the technical spirits and essential features thereof. Therefore, the above-described embodiments are exemplary in all aspects, and should be construed not to be restrictive.

What is claimed is:

1. A food information providing apparatus comprising:
an optical spectrum obtaining unit configured to obtain an optical spectrum of a food which a user is to take;
a depth image obtaining unit configured to obtain a depth image of the food; and
a controller configured to identify a kind of the food based on the optical spectrum of the food,
wherein the controller sets a figure with reference to a food area detected from the depth image of the food, and calculates at least one of a volume, a total calorie, and a suitable intake calorie of the identified food based on the set figure.

2. The food information providing apparatus of claim 1, wherein the controller converts image coordinates of pixels included in the detected food area to world coordinates, and sets one or more figures such that the one or more figures include the food area converted to the world coordinates.

3. The food information providing apparatus of claim 1, wherein the controller converts image coordinates of pixels included in the detected food area to world coordinates, and sets one or more figures in the food area converted to the world coordinates.

4. The food information providing apparatus of claim 1, wherein the figures include three-dimensional figures including at least one of a sphere, a cone, a cylinder, and a hexahedron.

5. The food information providing apparatus of claim 1, further comprising:
a storage configured to store a food information table,
wherein the food information table includes at least one of kinds of foods, types of natural optical spectrums of the foods, and calories per unit volume of the foods.

6. The food information providing apparatus of claim 5, wherein the controller obtains the calorie per unit volume of the identified food from the food information table, and calculates a total calorie of the identified food based on the calorie per unit volume of the identified food and the volume of the identified food.

7. The food information providing apparatus of claim 5, wherein the controller calculates a suitable intake calorie for the identified food with reference to at least one of current body information of the user and body information that is targeted by the user.

8. The food information providing apparatus of claim 1, further comprising:
an output unit configured to output at least one of a kind of the identified food, a total calorie of the identified food, and a suitable intake calorie of the identified food in at least one form including a letter, an image, and a voice.

9. The food information providing apparatus of claim 1, wherein the controller detects a food area corresponding to the food from the image obtained by photographing the food, and highlights and displays an area of the detected food area, which corresponds to the suitable intake calorie.

10. A method for providing food information, the method comprising:
obtaining an optical spectrum of a food which a user is to take;
obtaining a depth image of the food;
identifying a kind of the food based on the optical spectrum of the food; and
setting a figure with reference to a food area detected from the depth image of the food, and calculating at least one of a volume, a total calorie, and a suitable intake calorie of the identified food based on the set figure.
